(19) United States
(12) Patent Application Publication
(10) Pub. No.: US 2005/0053954 A1
(43) Pub. Date: Mar. 10, 2005

(54) Multicomponent protein microarrays

(76) Inventors: John D. Brennan, Dundas (CA); Nicholas Rupcich, Oakville (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/698,492

(22) Filed: Nov. 3, 2003

Related U.S. Application Data
(60) Provisional application No. 60/422,892, filed on Nov. 1, 2002.

(51) Int. Cl. C12Q 1/68; C12M 1/34

(52) U.S. Cl. 435/6; 435/287.2

(57) ABSTRACT

The present invention involves a multicomponent protein microarray comprising two or more components of a protein-based system entrapped within spots of a biomolecule compatible matrix arranged on a surface. Also included are methods of using the microarray for multicomponent analysis along with kits and machinery comprising the microarray.

### FIELD OF THE INVENTION

The present invention relates to protein microarrays, in particular protein microarrays wherein each microarray element contains two or more components, for use, for example, for the analysis of coupled reaction assays or of modulators of protein-molecule interactions.

### BACKGROUND TO THE INVENTION

Historically, enzyme activity and inhibition studies were conducted by focusing on a single protein at a time, resulting in time consuming and costly efforts. The recent development of multianalyte detection formats has allowed researchers to perform large-scale DNA and proteomic analyses. The technology of the microarray has the advantage of being scalable, and their ordered nature lends itself to high-throughput screening using robotics and analytical imaging techniques. Microarrays have revolutionized methods for high throughput analysis for several DNA experiments; including gene expression, sequence recognition (hybridization) and other DNA binding events. Extension of this technology to protein microarrays has recently been described, and several recent reviews have detailed the use of microarrays for applications such as screening antibody libraries and evaluation of protein-protein interactions.^{2,3,4,5,6,7}

Several immobilizations techniques and surface modification techniques have been employed in an attempt retain the activity of proteins immobilized onto surfaces. The three main techniques for protein immobilization on microarrays are covalent attachment, affinity capture and coupling to a hydrogel composed of an acrylamide polymer with additives which enhance protein binding.⁸ However, each of these methods has limitations.⁹ Covalent attachment of proteins to chemically activated surfaces (e.g. aldehyde, epoxy, active esters)^{10,11,12} or via biomolecular interactions (e.g. streptavidin-biotin, His-tag-nickel chelates)^{13,14} at the slide surface provides a surface that is accessible to external solutions to allow assessment of protein-protein or other biomolecular interactions. However, these immobilization methods can result in improper orientation of the protein's active site and monolayer coverage of the surface, which limits signal-to-noise levels, and decreases protein stability with the introduction of an artificial linker. Affinity capture methods require the expression of several recombinant proteins (e.g. hexahistidine or glutathione S transferase fusion protein) and/or capture agents (e.g. aptamers or antibodies) and still suffer from the inability to immobilize these proteins in an active form due to dehydration. Furthermore, this method is limited to soluble proteins in most cases. Recent advances based on the use of protein-binding ligands (monoclonal antibodies, protein aptamers or nucleic acid aptamers) to capture proteins at the slide surface can overcome some of these limitations, but requires a time consuming and costly screening process to discover the specific ligand needed for each protein.¹⁵ Another form of immobilization of molecules within a matrix is via physical entrapment. ^{16,17,18,19,20}

Another further serious drawback of all of the above methods is that they are designed to allow immobilization of only a single component per array element (i.e., one type of protein per spot), although it is possible to immobilize two proteins in a spot if the two proteins have affinity for one another. Immobilization of proteins with non-protein based species, such as polymers or fluorophores, or the immobilization of multiple enzymes involved in coupled catalytic reactions is not amenable to these immobilization methods.

There remains a need for a system for microarraying multiple component protein interactions that will preserve the proteins' functions and allow for high density arrays in much the same way that researchers have been able to array nucleic acids.

### SUMMARY OF THE INVENTION

A new class of protein microarray that is based on co-entrapment of multiple components within a single array element has been developed. The co-entrapment was based on immobilization of two enzymes or an enzyme and fluorescent reporter molecule within a sol-gel-derived microspot that is formed by pin-printing of the sol-gel precursors onto a microscope slide. In another example, a protein-peptide interaction has been microarrayed and examined for its ability to be disrupted by a denaturant.

The microarraying of a coupled two enzyme reaction involving glucose oxidase and horseradish peroxidase along with the fluorogenic reagent Amplex Red allowed for “reagentless” fluorimetric detection of glucose. A second system involving the detection of urea using co-immobilized urease and fluorescein dextran was demonstrated based on the pH induced change in fluorescein emission intensity upon production of ammonium carbonate. In both cases, it was demonstrated that the changes in intensity from the array were time-dependent, consistent with the enzyme-catalyzed reaction. The rate of intensity change was also found to be dependent on the concentration of analyte added to the array, showing that such arrays can be useful for quantitative multianalyte biosensing.

A third system involving protein-peptide interactions consisted of rhodamine-labelled calmodulin (CaM) and rhodamine-labelled mellitin, and was based on a slightly different fluorescence-based screening method utilizing these same biomolecules entrapped in sol-gel derived monoliths. In the absence of antagonists or denaturants (such as guanidine hydrochloride), these two species exist in a complex that brings the two rhodamine labels into close proximity, resulting in self-quenching and thus a low fluorescence signal. Upon addition of the denaturant guanidine hydrochloride the complex was dissociated, resulting in separation of the two probes and a resultant enhancement in fluorescence intensity. Washing of the array resulted in recovery of the intact complex, and hence a lowering of the fluorescent signal, indicating that such a configuration is reversible. Addition of non-antagonists such as benzamidine (negative control) resulted in no changes in intensity above that obtained for CaM alone.

The above experiments show the advantage of sol-gel microarrays for the entrapment of multiple species.

Accordingly, the present invention relates to a microarray comprising one or more spots of a biomolecule-compatible matrix having two or more components of a protein-based system entrapped therein, wherein the one or more spots are adhered to a surface.

Also included within the scope of the present invention is a method of preparing a microarray comprising:
(a) combining two or more components of a protein-based system with one or more biomolecule-compatible matrix precursor solutions; and
(b) applying the combination of (a) to a surface in a microarray format.

In a further embodiment of the invention, the method of preparing a microarray further comprises:
(c) allowing the combination of (a) to gel on the surface.

The present invention further relates to a method of performing multi-component assays comprising:
(a) obtaining one or more biomolecule compatible microarrays comprising a matrix having two or more components of a protein-based system entrapped therein;
(b) exposing the one or more biomolecule-compatible microarrays to one or more test substances; and
(c) detecting one or more changes in the protein-based system.

The method and microarray of the present invention may be used for any number of applications. For example, the multicomponent microarray of the present invention may be used for high-throughput drug screening, as multianalyte biosensors and as research tools for the discovery of new biomolecular interactions or antagonists or effectors of such interactions, or for the elucidation of protein function.

The invention also includes biosensors, micro-machined devices and medical devices comprising the multicomponent microarray of the present invention.

The present invention also includes relational databases containing data obtained using the microarray of the present invention.

The present invention further includes kits combining, in different combinations, the microarrays, reagents for use with the arrays, signal detection and array-processing instruments, databases and analysis and database management software above.

Yet another aspect of the present invention provides a method of conducting a target discovery business comprising:
(a) providing one or more assay systems for identifying test substances by their ability to effect one or more protein based systems, said assay systems using one or more of the microarrays of the invention;
(b) (optionally) conducting therapeutic profiling of the test substances identified in step (a) for efficacy and toxicity in animals; and
(c) licensing, to a third party, the rights for further drug development and/or sales or test substances identified in step (a), or analogs thereof.

The sol-gel entrapment method of protein immobilization for the production of protein microarrays has benefits beyond those of covalent or biomolecular attachment. Proteins remain active and hydrated in a matrix which has extensive functional derivitability, which until now has been explored very little in terms of biocompatibility. The demonstrations illustrated hereinbelow display the extreme potential of sol-gel protein microarrays as ultra-high throughput devices for the screening of several multicomponent biological interactions.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows images of a 5×5 array of co-immobilized urease and fluorescein dextran, including both positive and negative controls. Rows 1 and 5 contain both urease and fluorescein dextran, row 4 is sodium silicate only and acts as a blank, row 3 contains on fluorescein dextran and acts as a pH control and row 2 contains the enzyme acetylcholinesterase and fluorescein dextran and acts as a negative control. Addition of urea results in an enzymatic reaction creating a shift toward more basic pH values, producing an increase in emission intensity from 1a to 1b only in rows 1 and 5. Relative changes in intensity are shown in the figure. All spots are 100 μm wide.

FIG. 2 Panel (A): Is a graph showing average rates of intensity change with time for the urease microarray as a function of urea concentration (0.1 to 25 mM) introduced to the array. Panel (B): Is a graph showing concentration response for the addition of urea to the urease microarray.

FIG. 3 is a graph showing average changes in the rate of hydrolysis of 20 mM urea as a result of differing levels of the inhibitor thiourea introduced to the microarray.

FIG. 4 shows a 5×5 microarray of glucose oxidase/horseradish peroxidase co-immobilized in sol-gel derived glass. Columns 1 and 5 contain GOx/HRP co-immobilized with Amplex Red (coupled reaction site), column 2 contains only buffer and Amplex Red and acts as a negative control, column 3 contains GOx/HRP and glucose along with partially reacted Amplex Red, and acts as a positive control. Column 4 contains only GOx and Amplex Red and serves as a negative control. The first panel is before the addition of glucose (only column 3 is fluorescent owing to the presence of resorufin). The middle panel is one minute after addition of glucose and the third panel is 12 min after glucose addition, showing the time dependence of the enzyme catalyzed reaction. All spots are 100 μm wide.

FIG. 5 is a graph showing the kinetic response of the GOx/HRP array as a function of glucose concentration. PANEL A: Average change in fluorescence intensity with time at various glucose concentrations. PANEL B: Initial slope of fluorescence response vs. glucose concentration.

FIG. 6 contains images of an array comprised of co-entrapped calmodulin and melittin before and after exposure to a 20:1 molar ratio of guanidine hydrochloride:CaM (positive control, row 1), fluphenazine:CaM (test system, row 2). Columns 1 & 5 contain the protein—protein interaction between CaM and Mellitin. Both of which are labelled with rhodamine. Columns 2 & 4 are blank and contain only buffer. Column 3 contains CaM—Rhodamine alone and acts as a positive control. Upon addition of GdHCl (2M) to the top of the array and imaging every 20 s, the CaM-Mel columns increased in fluorescence over 2-fold, while the positive control increased slightly initially but flat-lined quickly.

FIG. 7 is a graph showing the increase in fluorescence intensity over time upon guanidine hydrochloride (DgHCl) addition to the CaM-Mel interaction for both the test sample and positive control.

### DETAILED DESCRIPTION OF THE INVENTION

To construct protein microarrays, it is desirable to immobilize the protein samples on a solid support. In order to study a protein in its active form, it is advantageous for this immobilization to preserve the folded conformation of the protein. Previous methods of protein immobilization can have deleterious effects on protein activity and are not amenable to the co-immobilization of multiple components of a protein-based system. These limitations are overcome in the present invention by entrapping the multiple components of a protein-based system within the confines of a bio-molecule compatible matrix. In this manner, the protein and other components can freely move within an element of the matrix and, therefore maintain their activity.

An example of a sol-gel encapsulation technique for the preparation of protein microarrays utilizing co-entrapment of either a coupled enzyme reaction involving glucose oxidase (Gox) and horseradish peroxidase (HRP), or of urease with fluorescein-dextran, has been developed. In the former case, the product of the coupled reaction reacted with Amplex Red to produce the fluorescent compound resorufin, which was used to develop a fluorescence readout. In the latter case, the ammonium carbonate produced by the urease-catalyzed hydrolysis of urea produced a shift toward basic pH which resulted in enhanced fluorescence from fluorescein-labelled dextran. As shown in the case of glucose oxidase, it was possible to design the microarrays with all necessary controls built into the microarray so that parallel acquisition of data from samples, blanks and control samples could be obtained simultaneously. Alternatively, separate arrays could be used for samples and blanks. It was also shown that the enzyme arrays could be read in a time-dependent manner to allow concentration-dependent assays of glucose or urea based on changes in fluorescence intensity with time, leading to the potential for quantitative multianalyte biosensing using such microarrays. Detection of an inhibitor of the urease-urea reaction has also been demonstrated, showing that such microarrays can find use in high-throughput drug-screening.

Another example of a sol-gel derived microarray involving a protein-peptide interaction has been developed consisting of rhodamine-labelled calmodulin (CaM) and rhodamine-labelled melittin co-entrapped in a sodium silicate derived sol-gel. In the absence of antagonists, these two species exist in a complex that brings the two rhodamine labels into close proximity, resulting in a self-quenching dimer and thus a low fluorescence signal. Upon addition of the antagonist guanidine hydrochloride at a 20:1 molar ratio (with respect to CaM) the complex was dissociated, resulting in separation of the two probes and a resultant enhancement in fluorescence intensity. Washing of the array resulted in recovery of the intact complex, and hence a lowering of the fluorescent signal, indicating that such a configuration is reversible.

Accordingly, the present invention relates to a microarray comprising one or more spots of a biomolecule-compatible matrix having two or more components of a protein-based system entrapped therein, wherein the one or more spots are adhered to a surface. In an embodiment of the invention, the one or more spots of the biomolecule-compatible matrix are arranged in a spatially defined manner on the surface.

As used herein, the term “spatially defined” means that the one or more spots of biomolecule-compatible matrix are arranged in a pre-determined pattern on a surface. Typically the pattern is ordered to facilitate the detection of any activity readout. In embodiments of the invention, the spots are arranged in parallel rows and columns. In further embodiments of the invention, the one or more spots are arranged in a manner such that their positions are known or are determinable.

As used herein, the term “entrapped” means that the components of the protein-based system are physically, electrostatically or otherwise confined within the nanometer-scale pores of the biomolecule-compatible matrix. In an embodiment of the invention, the proteins do not associate with the matrix, and thus are free to rotate within the solvent-filled pores. In a further embodiment of the invention, the entrapped protein is optionally further immobilized through electrostatic, hydrogen-bonding, bioaffinity, covalent interactions or combinations thereof, between one or more of the protein components and the matrix. In a specific embodiment, the entrapment is by physical immobilization within nanoscale pores.

The term “adhered” as used herein means to be sufficiently fixed to the surface so that the matrix is not washed off under typical washing and/or reactions conditions.

The term “spots” as used herein means a defined area. The spot may be any shape and does not necessarily have to be circular.

By “biomolecule-compatible” it is meant that the matrix either stabilizes proteins and/or other biomolecules against denaturation or does not facilitate denaturation. The term “biomolecule” as used herein means any of a wide variety of proteins, enzymes, organic and inorganic chemicals, other sensitive biopolymers including DNA and RNA, and complex systems including whole or fragments of plant, animal and microbial cells that may be entrapped in the matrix.

In embodiments of the invention, the biomolecule-compatible matrix is a sol-gel. In particular, the sol-gel is prepared using biomolecule-compatible techniques, i.e. the preparation involves biomolecule-compatible precursors and reaction conditions that are biomolecule-compatible. In another embodiment of the invention, the sol-gel matrix is conducive to maintaining the viability of the entrapped protein(s). For example, it adheres well to the surface and it resists cracking and/or washing away upon enduring repetitive wash cycles. In a further embodiment of the invention, the biomolecule-compatible sol gel is prepared from a sodium silicate precursor solution. In still further embodiments, the sol gel is prepared from organic polyol silane precursors. Examples of the preparation of biomolecule-compatible sol gels from organic polyol silane precursors are described in inventor Brennan's co-pending patent applications entitled “Polyol-Modified Silanes as Precursors for Silica”, PCT patent application S.N. PCT/CA03/00790, filed on Jun. 2, 2003 and corresponding U.S. patent application filed on Jun. 2, 2003; and “Methods and Compounds for Controlling the Morphology and Shrinkage of Silica Derived from Polyol-Modified Silanes”, PCT patent application S.N. PCT/CA03/01257, filed Aug. 25, 2003 and corresponding U.S. patent application filed on Aug. 25, 2003, the contents of all of which are incorporated herein by reference. In specific embodiments of the invention, the organic polyol silane precursor is prepared by reacting an alkoxysilane, for example tetraethoxysilane (TEOS) or tetramethoxysilane (TMOS), with an organic polyol. In an embodiment, the organic polyol is selected from sugar alcohols, sugar acids, saccharides, oligosaccharides and polysaccharides. Simple saccharides are also known as carbohydrates or sugars. Carbohydrates may be defined as polyhydroxy aldehydes or ketones or substances that hydroylze to yield such compounds. The organic polyol may be a monosaccharide, the simplest of the sugars, or a carbohydrate. The monosaccharide may be any aldo- or keto-triose, pentose, hexose or heptose, in either the open-chained or cyclic form. Examples of monosaccharides that may be used in the present invention include one or more of allose, altrose, glucose, mannose, gulose, idose, galactose, talose, ribose, arabinose, xylose, lyxose, threose, erythrose, glyceraldehydes, sorbose, fructose, dextrose, levulose and sorbitol. The organic polyol may also be a disaccharide, for example, one or more of, sucrose, maltose, cellobiose and lactose. Polyols also include polysaccharides, for example one or more of dextran, (500-50,000 MW), amylose and pectin. In embodiments of the invention the organic polyol is selected from one or more of glycerol, sorbitol, maltose, trehelose, glucose, sucrose, amylose, pectin, lactose, fructose, dextrose and dextran and the like. In embodiments of the present invention, the organic polyol is selected from glycerol, sorbitol, maltose and dextran. Some representative examples of the resulting polyol silane precursors suitable for use in the methods of the invention include one or more of diglycerylsilane (DGS), monosorbitylsilane (MSS), monomaltosylsilane (MMS), dimaltosylsilane (DMS) and a dextran-based silane (DS). In embodiments, the polyol silane precursor is selected from one or more of DGS and MSS.

In further embodiments of the invention, the biomolecule-compatible matrix precursor is selected from one or more of functionalized or non-functionalized alkoxysilanes, polyolsilanes or sugarsilanes; functionalized or non-functionalized bis-silanes of the structure (RO)₃Si—R′—Si(OR)₃, where R may be ethoxy, methoxy or other alkoxy, polyol or sugar groups and R′ is a functional group containing at least one carbon (examples may include hydrocarbons, polyethers, amino acids or any other non-hydrolyzable group that can form a covalent bond to silicon); functionalized or non-functionalized chlorosilanes; and sugar, polymer, polyol or amino acid substituted silicates.

In yet another embodiment of the present invention, the biomolecule compatible matrix further comprises an effective amount of one or more additives. In embodiments of the invention the additives are present in an amount to enhance the mechanical, chemical and/or thermal stability of the matrix and/or system components. In an embodiment, the mechanical, chemical and/or thermal stability is imparted by a combination of precursors and/or additives, and by choice of aging and drying methods. Such techniques are known to those skilled in the art. In further embodiments of the invention, the additives are selected from one or more of humectants and other protein stabilizing agents (for e.g. osmolytes). Such additives include, for example, one or more of organic polyols, hydrophilic, hydrophobic, neutral or charged organic polymers, block or random co-polymers, polyelectrolytes, sugars (natural or synthetic), and amino acids (natural and synthetic). In embodiments of the invention, the one or more additives are selected from one or more of glycerol, sorbitol, sarcosine and polyethylene glycol (PEG). In further embodiments, the additive is glycerol.

In a particular embodiment of the invention biocompatible matrix is a silica based glass prepared from, for example, a silicon alkoxide, alkylated metal alkoxide or otherwise functionalized metal alkoxide or a corresponding metal chloride, silazane, polyglycerylsilicate, diglycerylsilane or other silicate precursor, optionally in combination with additives selected from one or more of any available organic polymer, polyelectrolyte, sugar (natural or synthetic) or amino acids (natural and non natural).

The term “protein”, as used herein, refers to proteins, polypeptides, and peptides of any size, structure, or function. Typically, a protein will be at least three amino acids long, specifically at least 10 amino acids in length, more specifically at least 25 amino acids in length, and most specifically at least 50 amino acids in length. Proteins may also be greater than 100 amino acids in length. A protein may refer to a full-length protein or a fragment of a protein. Proteins may contain only natural amino acids or may contain non-natural amino acids and/or amino acid analogs as are known in the art. Also, one or more of the amino acids in the protein may be modified, for example, by the addition of a chemical entity such as a carbohydrate group, a hydroxyl group, a phosphate group, a famesyl group, an isofarnesyl group, a myristoyl group, a fatty acid group, functionalization, or other modification. The protein may also be a single molecule or may be a multi-molecular complex comprising proteins, lipids, RNA, DNA, carbohydrates, or other molecule. The protein may be naturally occurring, recombinant, or synthetic, or any combination of these. The protein may also be comprised of a single subunit or multiple subunits, and may be soluble or membrane-associated.

Examples of proteins that may be used in the present invention include, but are not limited to, enzymes (e.g., proteases, kinases, synthases, synthetases, nucleozymes), extracellular matrix proteins (e.g., keratin, elastin, proteoglycans), receptors (e.g., LDL receptor, amino acid receptors, neurotransmitter receptors, hormone receptors, globular protein coupled receptors, adhesion molecules), signaling proteins (e.g., cytokines, insulin, growth factors), transcription factors (e.g., homeodomain proteins, zinc-finger proteins), transport proteins (i.e., hemoglobin, human serum albumin), regulatory proteins (i.e., calmodulin, glucose binding protein) and members of the immunoglobulin family (e.g., antibodies, IgG, IgM, IgE).

The protein-based system may be any system involving a protein and any other component. In an embodiment of the invention, the microarray is used to assay a certain activity in one or more proteins in a system, for example, catalytic activity, an ability to bind another protein or an ability to bind a nucleic acid or small molecule. In embodiments of the present invention, the components of a protein-based system include two or more enzymes involved in a coupled catalytic reaction, or one or more proteins and one or more chemical entities, for example one or more reagents that may be used to detect the activity of the protein(s). The two or more components of the protein-based system may or may not have affinity for one another. The protein-based system may also include two or more separate protein-based reactions with no cross-reactivity. Each protein-based reaction may be comprised of a single protein or a multicomponent system.

In further embodiments of the present invention, the one or more components of a protein-based system include: two proteins or a protein and an aptamer, which form a complex for screening of potential ligands; a protein-membrane complex for screening of modulators of membrane bound receptors; or immobilzation of multicomponent protein:DNA aptamer complexes for sensing of biomarkers. Furthermore, the invention includes the case where the protein and aptamer or DNA or RNA enzyme are co-entrapped so that the aptamer or DNAzyme/RNAzyme provide a signal that responds to a protein-based reaction (i.e., detection of product from an enzyme-substrate reaction, or allosteric control of catalysis wherein the nucleozyme can bind to one conformation of a protein but not another, and is active only in one form (bound or unbound)).

The term “surface” refers to any solid support to which biomolecule compatible matrixes can be printed. In an embodiment of the invention, the surface is a substantially planar surface, for example a slide, the distal end of a fiber optic bundle, a suitably machined light emitting diode, a planar waveguide or any other surface onto which sub-millimeter elements can be placed. With proper calibration of the arraying system, deposition onto curved surfaces may also be done, allowing coating of lenses, microwells within microwell plates and other surfaces. The surface is typically a solid support made of, for example, glass, plastic, polymers, metals, ceramics, alloys or composites. In embodiments of the invention, the surface is a glass microscopic slide which has been cleaned to remove any organic matter and any adsorbed metal ions. Further modification of the glass surface with for example, aminopropyltriethoxysilane (APTES) or glycidoxyaminopropyltrimethoxysilane (GPS), provides the glass slide with an improved adhesion with the sol-gel matrix due to stronger hydrogen bonding and acid-base interactions between their amino groups and the silicate. This results in matrix spots which do not spread once they are printed and promotes spot uniformity in size and shape.

Also included within the scope of the present invention is a method of preparing a microarray comprising:
(a) combining two or more components of a protein-based system with one or more biomolecule-compatible matrix precursor solutions; and
(b) applying the combination of (a) to a surface in a microarray format.

In a further embodiment of the invention, the method of preparing a microarray further comprises, in order:
(c) allowing the combination of (a) to gel on the surface.

The term “gel” as used herein means to lose flow.

The protein microarrays of the present invention may be prepared by combining the one or more matrix precursor solution(s) with one or more solutions comprising the two or more components of a protein-based system, with the precursor(s) and system components being combined in any suitable ratio, for example any ratio ranging from about 1:10 up to about 10:1. In an embodiment of the invention, the precursor(s) and system components are combined in approximately a 1:1 ratio. The resulting combination is then applied, for example in a spatially-defined manner, onto a surface using any known technique, for example by a commercially available automated arrayer, such as an automated pin-printer, an ink-jet electrospray deposition system or a microcontact printing (stamping) technique.

The size of the spatially defined spots can be controlled to any suitable range, for example, having a range of 50 to 500 μm, as can the spacing between them, for example having a range of 0 μm to the maximum width of the printing surface. In an embodiment of the invention, the spots are on the order of 100 μm in diameter and are 150-200 μm apart.

In further embodiments of the invention, the two or more components of a protein-based system and suitable biomolecule-compatible precursor solution(s) are combined with an effective amount of one or more additives. In embodiments of the invention the additives are present in an amount effective to impart mechanical, chemical and/or thermal stability to the matrix. In embodiments of the invention, the additives are selected from one or more of humectants and other protein stabilizing agents (for e.g. osmolytes). Such additives include, for example, one or more of polyols, hydrophilic, hydrophobic, neutral or charged organic polymers, block or randon co-polymers, polyelectrolytes, sugars (natural or synthetic), and amino acids (natural and synthetic). In embodiments of the invention, the one or more additives are selected from one or more of glycerol, sorbitol, sarcosine and polyethytlene glycol (PEG). For example, the one or more additives may include an effective amount, for example in the range of 0.5% to 50% (v/v), more specifically 5-30% (v/v), of a humectant or other protein stabilizing agent (e.g., osmolytes), for example glycerol or polyethylene glycol, to inhibit evaporation and/or stabilize the entrapped protein (i.e. to keep the protein hydrated and in an active state). The humectant may also act as a biocompatible molecule whose presence stabilizes the entrapped protein or prevents its denaturation. When the precursor solution comprises an organic polyol-derived silane, for example DGS or MSS, it is an embodiment of the invention that an effective amount, for example about 0.5%-50%, more specifically about 5%-35%, more specifically about 15%-30%, of a humectant, for example glycerol, be used.

Once the microarray is formed on the surface, it may be exposed to one or more test substances that are, for example, candidates as substrates of the protein and/or modulators of the protein(s), and the ability of the one or more proteins to act on these substances assayed. Accordingly, the present invention further relates to a method of performing multi-component assays comprising:
(a) obtaining one or more biomolecule compatible microarrays comprising a matrix having two or more components of a protein-based system entrapped therein;
(b) exposing the one or more biomolecule-compatible microarrays to one or more test substances; and
(c) detecting one or more changes in the protein-based system.

In an embodiment of the present invention, the systems involve coupled enzyme reactions. In this embodiment, the protein-based system may involve a first enzyme, the activity of which is detected or monitored by the conversion by a second enzyme of its reaction product into a compound that is detectable, for example by fluorescence, and the formation of that detectable product is monitored. In this example, the two enzymes are entrapped within the biomolecule-compatible matrix and the matrix formed into a microarray. The microarray may then be treated with the substrate of the first enzyme and the formation of the product monitored. Optionally, the microarray may be treated with a combination of substrate and other test substances, for example small molecules, that may modulate the activity of the first enzyme. The effect of the potential modulators on the activity of the first enzyme may then be determined. In this manner, the microarray may be used for high-throughput screening (HTS) of potential modulators of the first enzyme. An example of this type of system is the Gox/HRP system as described in Example 2 hereinbelow. Either the first or second enzyme, or both, may be derived from either amino acids (natural or non-natural) or either ribonucleotides or deoxyribonucleotides, producing ribozymes or deoxyribozymes, respectively, collectively referred to as nucleozymes. Furthermore, the nucleozymes may be designed to produce a fluorescence response upon production of a product by the first enzyme reaction (as in the well-known riboreporter system), and thus may act as reporters of the enzyme-substrate reaction, or inhibition thereof. Clearly, such a method could be extended to include the case where more than two proteins are present, and could involve detection of loss of substrate or production of product, or inhibition thereof.

In further embodiments the activity of an enzyme may be monitored by the conversion of another chemical entity into a detectable product by a change in conditions upon reaction of the enzyme with its substrate. In this case, the enzyme and other chemical entity are entrapped within the biomolecule-compatible matrix and the matrix formed into a microarray. The microarray may then be treated with the substrate of the first enzyme and the formation of the product monitored. Once again, the microarray may optionally be treated with a combination of substrate and other test substances, for example small molecules, that may modulate the activity of the enzyme. The effect of the potential modulators on the activity of the first enzyme may then be determined. In this manner, the microarray may be used for high-throughput screening (HTS) of potential modulators of the enzyme. An example of this type of system is the urease/fluorescein dextran system as described in Example 1 hereinbelow.

In still further embodiments of the present invention, the protein-based system includes a receptor and the binding of potential modulators of the receptor are screened using a microarray of the present invention. The protein-based system may also be a complex of two or more proteins, or a protein and an aptamer, and the microarray may be used to screen for potential ligands that can bind to or effect the binding between these entities. In these latter two embodiments, the system or the compounds may be labelled, using for example a fluorescent or a radioactive label, to facilitate the detection of binding. In a specific embodiment of the above example, a small molecule or biomolecular modulator of protein function may compete with an aptamer or second protein for binding to the active site or an allosteric site on the primary protein. In such as case, the aptamer or secondary protein will act as a surrogate ligand to allow for high-throughput screening of protein-small molecule or protein-protein interactions using either competitive or displacement assays. Such assays can be used to examine kinase phosphorylation reactions, protein-protein/DNA/RNA/small molecule binding events or disruption of these bound systems using fluorescence reporting or other readout methods as described below.

The multicomponent microarrays can also be used to allow for simultaneous spatial and spectral discrimination of reactions. In one such embodiment, the protein-based system comprising two separate protein-based reactions (with no cross-reactivity) may be co-entrapped in a single array element (in this case each protein-based system may be comprised of a single protein or of a multi-component system). The first reaction will produce a signal that is either excited or detected at one wavelength, and the other reaction will produce a signal that is either excited or detected at a different wavelength that does not interfere with the first reaction. In this way, two or more reactions can be examined in the same microarray element simultaneously by employing two detection wavelengths. A person skilled in the art will appreciate that this concept can be extended to include the case where two or more different readout methods are used.

In a further embodiment of the present invention, the protein microarray includes one or more spots containing positive and/or negative controls. This may be done by preparing spots containing partial or no reaction starting materials (for negative controls) and/or all of the reaction starting materials, including the known substrates or ligands for the proteins/enzymes (positive control), on the same surface as the “test” spots. In one embodiment of the invention, the positive and/or negative controls are located in separate columns or rows adjacent to the “test” spots, however it is clear that any pattern of controls can be incorporated in the array or two or more arrays can be created where each different array can contain for example blanks, positive controls, negative controls etc. Accordingly, the method of performing multi-component assays according to the present invention further comprises comparing the change in the protein based system to a control, wherein a change in the protein based system upon exposure to one or test substances compared to the control is indicative of the effect of the one or more test substances on the protein based system.

The protein activity or binding interactions that are assayed using the methods of the present invention may be detected via any method known in the art including fluorescence, radioactivity, immunoassay, etc. (for more detail on these methods, please see Ausubel et al., eds., Current Protocols in Molecular Biology, 1987; Sambrook et al. Molecular Cloning: A Laboratory Manual, 2nd Ed., 1989; each of which is incorporated herein by reference). Imaging of the array using methods such as Raman scattering or other imaging methods is also possible.

The term “test substance” as used herein means any agent, including drugs, which may have an effect on the protein based system and includes, but is not limited to, small inorganic or organic molecules; peptides and proteins and fragments thereof; carbohydrates, and nucleic acid molecules and fragments thereof. The test substance may be isolated from a natural source or be synthetic. The term test substance also includes mixtures of compounds or agents such as, but not limited to, combinatorial libraries and extracts from an organism.

The method and microarray of the present invention may be used for any number of applications. For example, the multicomponent microarray of the present invention may be used for high-throughput drug screening, as multianalyte biosensors and as research tools for the discovery of new biomolecular interactions or for the elucidation of protein function.

The invention also includes kits, biosensors, micromachined devices and medical devices comprising the multicomponent microarray of the present invention.

The present invention also includes relational databases containing data obtained using the microarray of the present invention. The database may also contain sequence information as well as descriptive information about the protein system and/or the test compound. Methods of configuring and constructing such databases are known to those skilled in the art (see for example, Akerblom et al. U.S. Pat. No. 5,953,727).

As mention above, the present invention further includes kits combining, in different combinations, the microarrays, reagents for use with the arrays, signal detection and array-processing instruments, databases and analysis and database management software above. The kits may be used, for example, to determine the effect of one or more test compounds on a protein system and to screen known and newly designed drugs.

Databases and software designed for use with use with microarrays is discussed in Balaban et al., U.S. Pat. No. 6,229,911, a computer-implemented method for managing information, stored as indexed tables, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

Yet another aspect of the present invention provides a method of conducting a target discovery business comprising:
(a) providing one or more assay systems for identifying test substances by their ability to effect one or more protein based systems, said assay systems using one or more of the microarrays of the invention;
(b) (optionally) conducting therapeutic profiling of the test substances identified in step (a) for efficacy and toxicity in animals; and
(c) licensing, to a third party, the rights for further drug development and/or sales or test substances identified in step (a), or analogs thereof.

By assay systems, it is meant, the equipment, reagents and methods involved in conducting a screen of compounds for the ability to modulate one or more protein-bases systems using the method of the invention.

The following non-limiting examples are illustrative of the present invention:

### EXAMPLES

Materials and Methods

Chemicals. Urease (type IX from Jack Beans, 35,400 units.g⁻¹ solid), urea, thiourea, glycerol, acetylcholinesterase (AChE, Type VI-S from electric eel, 400 units.g⁻¹ solid) and Dowex 50×8-100 cation exchange resin were obtained from Sigma (St. Louis, Mo.). γ-aminopropylsilane (GAPS) derivatized glass microscope slides were purchased from Corning (Coming, N.Y.). Sodium silicate (SS, technical grade, 9% Na₂O, 29% silica, 62% water) was purchased from Fisher Scientific (Pittsburgh, Pa.). Fluorescein dextran (FD, 70,000 MW) and an Amplex Red glucose/glucose oxidase assay kit were obtained from Molecular Probes (Eugene, Oreg.). Water was purified with a Milli-Q Synthesis A10 water purification system. All other chemicals and solvents used were of analytical grade.

Preparation of Spotting Solutions: GOx and HRP were dissolved at concentration of 0.4 mg.mL⁻¹ (250 units.mg⁻¹) and 0.01 mg.mL⁻¹ (1000 units.mg⁻¹), respectively, in 50 mM sodium phosphate buffer, pH 7.4 to form the protein stock solutions. The Amplex Red reagent was made up to a stock concentration of 10 mM. Urease and fluorescein dextran were dissolved at concentrations of 2 mg.mL⁻¹ (35,400 units.g⁻¹) and 25 μM, respectively, in 50 mM Tris buffer containing 50 mM NaCl, pH 8 to form their respective stock solutions. GOx/HRP assay samples were prepared to a total volume of 50 μL by mixing 3 μL of each of the GOx and HRP stock solutions, 39 μL of sodium phosphate buffer and 5 μL of the Amplex Red dye solution. Negative and blank control samples were prepared in the same way except that phosphate buffer replaced the missing reagent. Positive control samples contained GOx and HRP as well as 15 μL of 100 μM D-glucose (in buffer) and only 24 μL of buffer. Urease/fluorescein dextran assay samples also had a total volume of 50 μL and were made up of 10 μL of fluorescein dextran stock and 40 μL of the urease stock solution. Similarly, the blank and positive control samples replaced the missing reagents with Tris buffer while the enzyme selectivity control was obtained by replacing urease with ACHE (0.01 mg.mL⁻¹) in Tris buffer.

The sodium silicate solution (SS) was prepared by diluting 5.8 g of sodium silicate in 20 mL of ddH₂O and immediately adding 10 g of the Dowex resin. The mixture was stirred for 30 seconds and then vacuum filtered through a Buckner funnel. The filtrate was then further filtered through a 0.45 μM membrane syringe filter to remove any particulates in the solution. Spotting solutions were formed by combining the precursor solution and the buffered enzyme sample solutions in a 1:1 (v/v) ratio in the well of a 96-well plate. Final reagent concentrations in the spotting solutions were as follows: 12 μg.mL⁻¹ GOx, 0.3 μg.mL⁻¹ HRP, 0.5 mM Amplex Red, 0.8 mg.mL⁻¹ urease, 4 μg.mL⁻¹ ACHE and 2.5 μM fluorescein dextran. The mixtures typically required at least 10 minutes to gel, minimizing the potential of the materials to gel within the printing pin.

Microarray Pin-Printing and Imaging. A Virtek Chipwriter Pro (Virtek Engineering Sciences Inc., Toronto, ON) robotic pinspotter equipped with a SMP 3 stealth microspotting pin (250 nL uptake, 0.6 nL delivery, Telechem Inc., Sunnyvale, Calif.) was used to print samples onto GAPS derivatized glass microscope slides from 96-well plates using a printhead speed of 16 mm.s⁻¹. Printing was done at room temperature with a relative humidity of approximately 50-70%. Fluorescence images of the microarrays were taken with an Olympus BX50 Microscope equipped with a Roper Scientific Coolsnap Fx CCD camera using a tunable multi-line argon ion laser source for excitation of fluorescein (488 nm) and resorufin (514 nm).

Enzyme Assays: All enzyme assays and inhibition studies were performed in 96 well plates using a TECAN Safire absorbance/fluorescence platereader operated in fluorescence mode, or on the microarray using time-dependent fluorescence intensity measurements. The enzymatic activity of free and entrapped GOx in 96 well plates was measured by adding 50 μL of a solution containing varying concentrations of glucose to the microtiter well and monitoring of the fluorescence emission at 590 nm for 20 minutes (in solution) or 45 minutes (for entrapped GOx) with excitation at 573 nm. For microarrays, 20 μL of a glucose solution was added to the top of the array, left for 20 seconds and then removed by gently blowing air over the surface, followed by monitoring of fluorescence emission over time. The removal of the glucose solution was done to reduce leaching of the Amplex Red probe, which was observed to occur after prolonged exposure of the array to aqueous solution. Images were acquired before the addition of glucose and then every 30 seconds for 30 minutes after the introduction of glucose using a 30 second integration time per image. For urease, activity and inhibition were measured by adding 100 μL of a solution containing a constant amount of urea (20 mM) in the presence of varying amounts of thiourea (0-100 mM) to the microtiter well and the fluorescence emission of the fluorescein dextran was monitored at 520 nm for 15 minutes (solution) or 45 minutes (entrapped). Microarrays containing urease and fluorescein dextran were first imaged after washing with distilled deionized water (ddH₂O, pH 5.1) to provide a constant baseline intensity response. Following this, 20 μL of a urea/thiourea solution was added to the top of the array, which was then covered with a coverslip to minimize solvent evaporation. The emission intensity of fluorescein dextran was measured every 20 seconds for 10 minutes using a 10 second integration time per image following the addition of the urea solution to the array. All samples were tested within 24 hours of being prepared.

For both enzymes studied the initial rate of change in fluorescence intensity was converted to a change in product concentration with time using calibration curves relating the emission intensity of fluorescein to the concentration of ammonium carbonate (for urease) or hydrogen peroxide (for GOx). The Michaelis constants (K_{M}) and catalytic rate constants (k_{cat}) for the enzymes were calculated by generating either double reciprocal (Lineweaver-Burk) plots relating (initial rate of product formation)⁻¹ to (substrate concentration)⁻¹ or Hanes-Wolff plots, and fitting these to a linear model. Inhibition constants (K_{I}) for urease were calculated by assessing the changes in the initial rate values for the enzyme in the presence of varying levels of inhibitor, according to the equation:

$${K}_{I} = \frac{[ I ]}{( {V}_{0} / {V}_{I} ) - 1}$$

where V₀ is the initial rate of substrate turnover in the absence of inhibitor, V_{I} is the initial rate of substrate turnover in the presence of inhibitor, and [I] is the concentration of inhibitor.

### Example 1

### Urease and Fluorescein-labelled Dextran

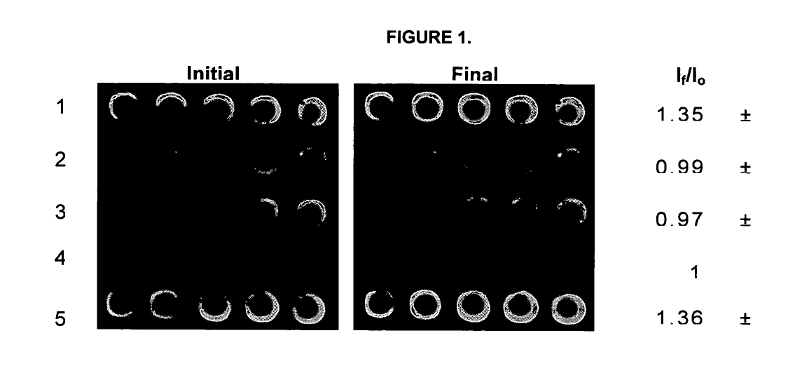

FIG. 1 shows images of a 5×5 microarray that were prepared for kinetic studies of immobilized urease. The array consisted of four different samples, composing a reagentless enzyme assay array that was suitable for sensing of both substrates and inhibitors. In this array, rows 1 and 5 contained urease that was co-immobilized with fluorescein labelled dextran. Also present in the array were a blank row consisting of only sodium silicate with buffer (negative control, row 2), a row containing only fluorescein dextran 70,000 MW as a pH selectivity control to avoid signals related to drifts in pH that were not based on the enzyme catalyzed reaction (row 3), and a row containing AChE with FD as a selectivity control (row 4). These controls ensured that the enhancement of intensity of any spots in the microarray following addition of urea were solely due to the activity and selectivity of the urease and were not due to drifts in pH or autohydrolysis of urea by the matrix. It is not clear why the arrays showed “donut” shaped intensity patterns. Brightfield imaging of the arrays showed that the sol-gel material was spotted in a hemispherical shape on the slide, and thus was not absent from the center of the spots. It is possible that the shape of the sol-gel spot resulted in a lensing effect that caused emission from the center of the spots to be directed away from the microscope objective.

The microarray was doped with a range of urea concentrations (0-25 mM) and then imaged in 30 second intervals over a period of 10 minutes to assess changes in the fluorescence intensity. Addition of urea results in an enzymatic reaction that creates a shift toward more basic pH values, producing an increase in emission intensity from the entrapped fluorescein dextran in the test array. The initial and final images of the microarray are shown in FIG. 1, along with the relative changes in intensity upon addition of urea. Only the spots containing both urease and the FD showed enhanced intensity following addition of urea (control elements showed no changes in emission intensity), indicating that the protein remained active and that selectivity for urea was retained within the sol-gel derived microarray elements.

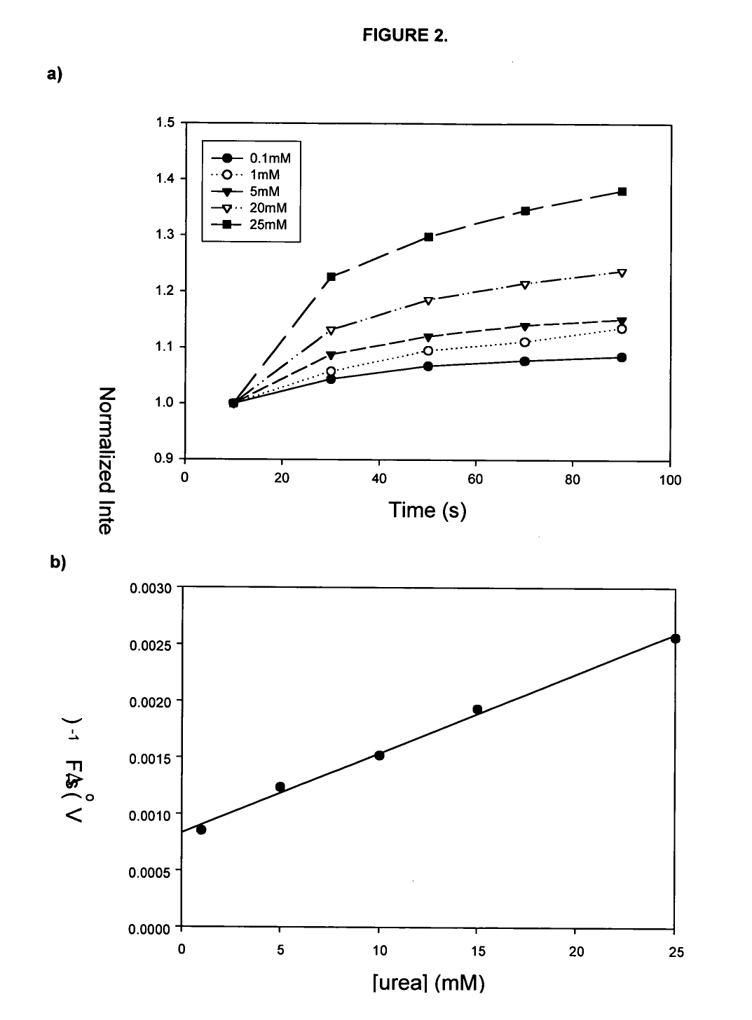

FIG. 2 shows the average rates of intensity change with time for the urease microarray as a function of urea concentration introduced to the array (Panel A), and the corresponding concentration response profile (Panel B). It is clear that concentration-dependent responses can be derived from microarrays, indicating that the changes in fluorescence intensity can be used for the determination of urea concentration. All data could be fit to Michaelis Menten kinetics, allowing for construction of Lineweaver-Burke or Hanes-Wolff plots to examine the K_{M} and k_{cat} values of urease on the microarray relative to the values obtained for free and entrapped urease as determined using a standard platereader. As shown in Table 1, the K_{M} values for urease were in all cases within a factor of two of the value in solution and are in good agreement with the literature value of 2.9 mM [²¹]. On the other hand, k_{cat} values were significantly lowered upon entrapment, with the value for the entrapped protein being up to 70-fold lower than in solution. Decreases in the catalytic rate constant for entrapped enzymes has been reported by several groups [²²,²³,²⁴,²⁵,²⁶], and is expected based on the tortuous path that must be taken to allow diffusion of small molecules through the porous network of the silica [²⁷]. The k_{cat} values were also lower than expected since the assays were performed at pH 5.1, which is shifted significantly away from the optimal pH of 7.4 for urease catalysis [²⁸,²⁹]. It is also possible that some of the urease had denatured upon entrapment, which would lead to a lowering of the catalytic rate constant. Even so, the data show that 1) concentration dependent fluorescence responses can be obtained on a microarray; 2) “reagentless” assays can be done conveniently on an array; and 3) entrapped enzymes on an array follow Michaelis-Menten kinetics.

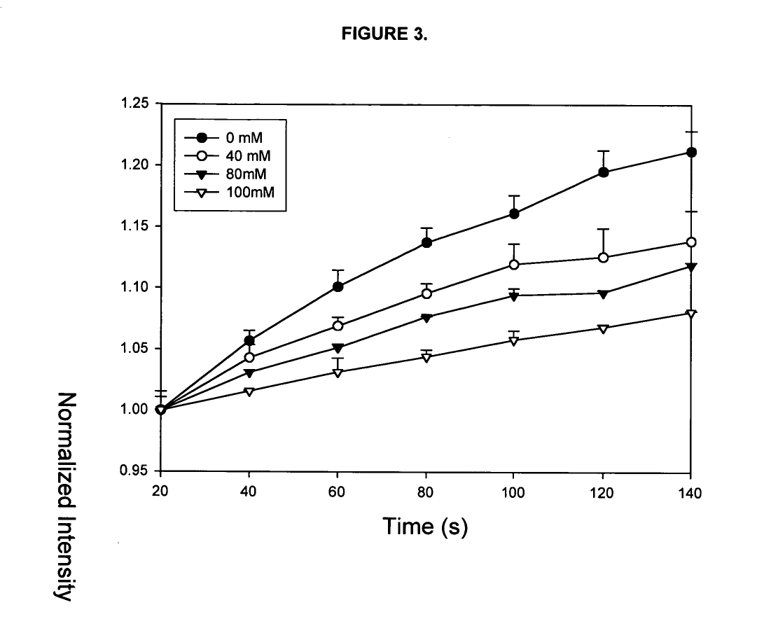

To examine whether entrapped enzymes on sol-gel derived microarrays were likely to be suitable for drug-screening, inhibition of urease on the array was examined. FIG. 3 shows the changes in signal magnitude upon addition of the different levels of the inhibitor thiourea to microarrays containing entrapped urease in the presence of a constant amount of urea. Both the rate of change of fluorescence intensity and the final fluorescence intensity decrease as the concentration of thiourea increase (note: control experiments indicated that thiourea did not quench the fluorescence of FD, thus the decrease in the intensity of FD is consistent with inhibition of urease). The inhibition constant (K_{I}) for thiourea was calculated for urease entrapped in bulk sodium silicate glass and deposited on the microarray using sodium silicate, and compared to the literature range of K_{I} values, 48-85 mM [21]. As shown in Table 1, the inhibition constants all fall within the literature range, indicating that inhibition of urease within the sol-gel derived microarray could be measured accurately. A recent study [26] demonstrated that one factor in determining the ability to accurately determine K_{I} values using entrapped enzymes is an absence of inhibitor partitioning between the solution and the entrapped enzyme. In sol-gel derived silica, partitioning generally results from electrostatic interactions between the anionic silica and charged analytes. Since neither urea nor thiourea are charged, the partitioning was not an issue. These results suggest that sol-gel based enzyme arrays will find use in high-throughput drug screening of multiple enzymes in a highly parallel fashion.

### Example 2

### Glucose Oxidase and Horseradish Peroxidase

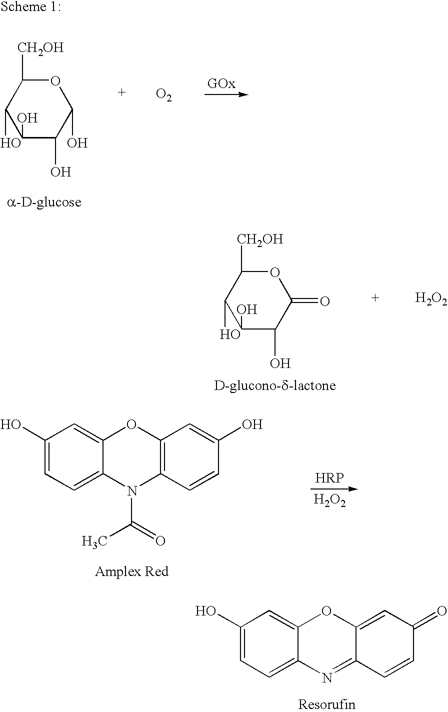

The second protein system that was examined in sol-gel derived microarrays was a more complex system, consisting of two proteins that undergo a coupled reaction. Glucose oxidase reacts with D-glucose to form D-gluconolactone and H₂O₂ (Scheme 1). In the presence of horseradish peroxidase, the H₂O₂ then reacts with the Amplex Red reagent in a 1:1 stoichiometry to generate the red fluorescent oxidation product, resorufin, as seen in Scheme 1. Resorufin has absorption and fluorescence emission maxima of approximately 563 nm and 587 nm, respectively, at pH>6 [³⁰,³¹]

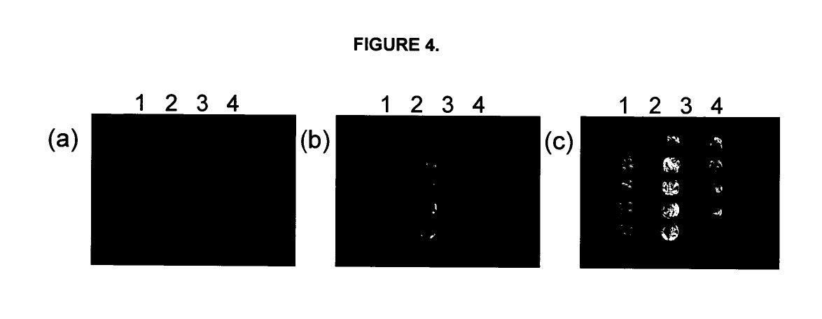

FIG. 4 shows a 5×5 array of Glucose Oxidase/Horseradish Peroxidase co-immobilized in sol-gel derived glass. Columns 1 and 5 contain GOx/HRP co-immobilized with Amplex Red (coupled reaction site). Column 2 contains only buffer and Amplex Red and acts as a negative control. Column 3 contains reacted GOx, HRP, glucose and partially reacted Amplex Red and acts as a positive control. Column 4 contains only GOx and Amplex Red and serves as a negative control. The first panel shows the array before the addition of glucose (only column 3 is fluorescent owing to the presence of resorufin). The middle panel shows the array one minute after the addition of glucose and the third panel shows the array 12 min after glucose addition. The only columns in the array that were illuminated after reacting for fifteen minutes were the positive control and the GOx/HRP sample (columns 1, 5 and 3 respectively in FIG. 4), showing the selectivity of the reaction on the microarray. Furthermore, the changes in intensity with time confirm the time-dependent nature of the assay, as expected for an enzyme catalyzed reaction. This example demonstrates the ability of co-entrapped enzymes to work together to produce an analyte-dependent fluorescent signal.

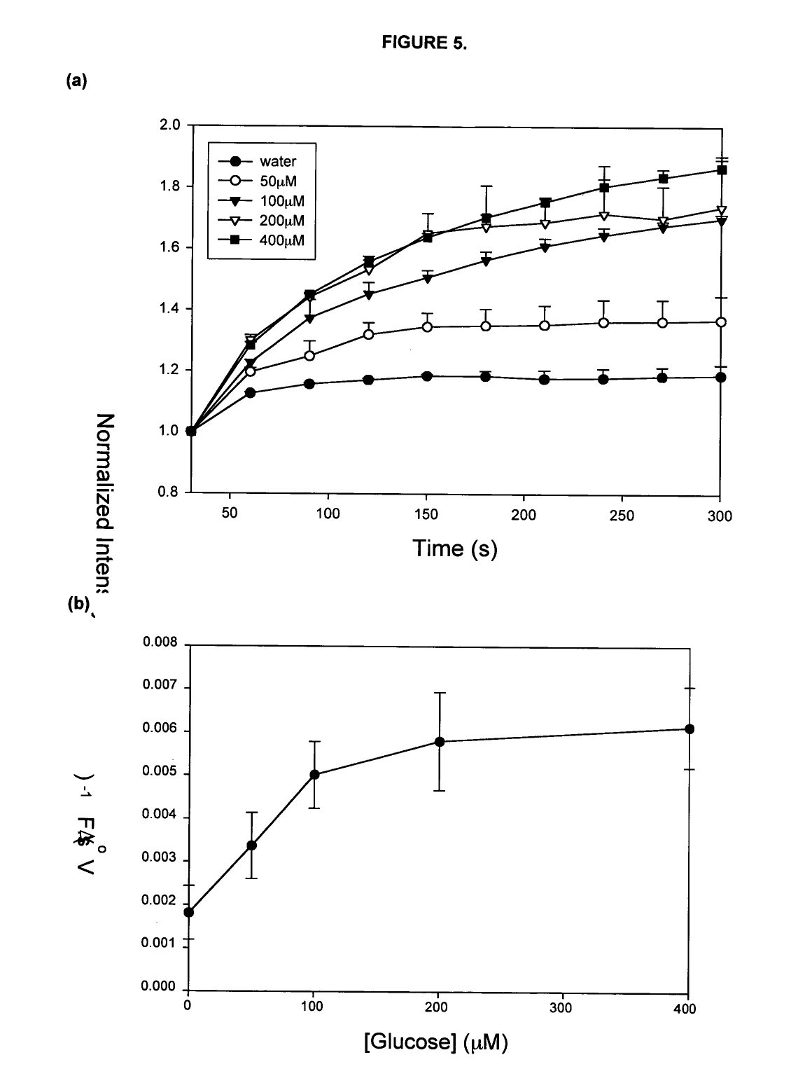

FIG. 5 shows the kinetic response as a function of glucose concentration introduced to the GOx/HRP array. Panel A shows the average changes in fluorescence intensity with time for the array elements containing both GOx and HRP as a function of glucose concentration. Increased levels of glucose up to 200 μM led to more rapid increases in fluorescence intensity with time, and to a higher plateau value of fluorescence intensity. Panel B shows the change in initial slope with glucose concentration, which follows the expected hyperbolic trend, showing the potential of the multicomponent enzyme microarrays for determination of substrate concentrations. Fitting of the data to the Michaelis-Menten equation provided the K_{M} and k_{cat} values shown in Table 1. The k_{cat} value of the entrapped enzyme was again lower than in solution, although in this case the k_{cat} values were within a factor of 20. As with urease, factors such as slow diffusion of glucose within the matrix, partial denaturation of either GOx or HRP, or pH effects may have played a role in reducing the k_{cat} value.

The K_{M} values obtained on the array were also within a factor of two of the values obtained in solution, although it is not clear why the K_{M} value of the entrapped enzyme increased when tested on the platereader but decreased on the array. More importantly, the K_{M} values were all in the micromolar range rather than the millimolar range, even in solution. For this reason the linear range of the array for glucose concentration was well outside of the physiologically relevant range (5-50 mM). However, this is a result of the nature of the Amplex Red sensitivity to H₂O₂, which results in a decrease in the apparent K_{M} for GOx [30].

### Example 3

### Calmodulin-Melittin Array

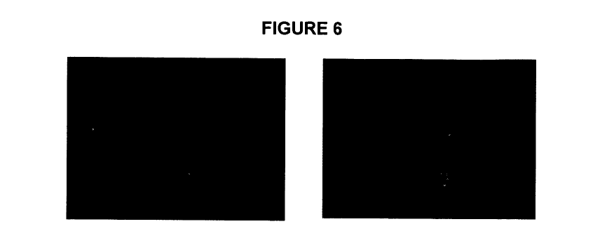

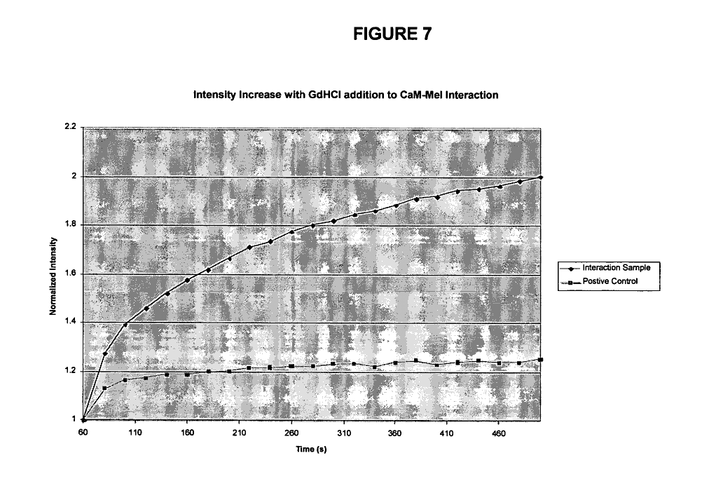

FIG. 6 shows an array comprised of co-entrapped calmodulin and melittin before and after exposure to a 20:1 molar ratio of guanidine hydrochloride:CaM. Columns 1 & 5 contain the protein—protein interaction between CaM and Mellitin. Both of which are labelled with rhodamine. Columns 2 & 4 are blank and contain only buffer. Column 3 contains CaM—Rhodamine alone and acts as a positive control. Upon addition of GdHCl (2M) to the top of the array and imaging every 20s, the CaM-Mel columns increased in fluorescence over 2-fold (Panel B), while the positive control increased slightly initially but reached a relatively low steady-state value quickly (see graph, FIG. 7)

While the present invention has been described with reference to the above examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

Full Citations for Documents Referred to in the Specification

[¹] Templin, M. F.; Stoll, D.; Schrenk, M.; Traub, P. C.; Vohringer, C. F.; Joos, T. O. Protein Microarray Technology. TRENDS in Biotechnology. 2002, 20-4, 160.
[²] Emili, A. Q.; Cagney, G. Large scale functional analysis using peptide or protein arrays. Nature Biotechnology. 2000, 18, 393.
[³] Zhu, H.; Snyder, M. Protein arrays and microarrays. Curr. Opin. Chemical Biology. 2001, 5, 40.
[⁴] Kodadek, T. Protein microarrays: prospects and problems. Chemistry & Biology. 2001, 8, 105.
[⁵] Walter, G.; Bussow, K.; Cahill, D.; Lueking, A.; Lehrach, H. Protein arrays for gene expression and molecular interaction screening. Curr. Opin. Biotechnology. 2000, 3, 298.
[⁶] Jenkins, R. E.; Pennington, S. R. Arrays for protein expression profiling: towards a viable alternative to two-dimensional gel electrophoresis? Electrophoresis. 2001, 1, 13.
[⁷] Cahill, D. J. Protein and antibody arrays and their medical applications. J Immunological Methods. 2001, 250, 81.
[⁸] Taylor, S.; Croker, K.; Weber, S. C. Gel Pad Arrays and Methods and Systems for Making Them. PCT Int. Appl. (1999) - WO9936576.
[⁹] Wilson, D. S.; Nock, S. Functional protein microarrays. Curr. Opin. Chemical Biology. 2001, 6, 81.
[¹⁰] Macbeath, G.; Schreiber, S. L. Printing Proteins as Microarrays for High-Throughput Function Determination. Science. 2000, 289, 1760.
[¹¹] Arenkov, P.; Kukhtin, A.; Gemmell, A.; Voloshchuk, S.; Chupeeva, V.; Mirzabekov, A. Protein Microchips: use for immunoassay and enzymatic reactions. Analytical Biochemistry. 2000, 278, 123.
[¹²] Zhu, H.; Klemic, J. F.; Chang, S.; Bertone, P; Casamayor, A.; Klemic, K. G.; Smith, D.; Gerstein, M.; Reed, M. A.; Snyder, M. Analysis of yeast protein kinases using protein chips. Nat Genet. 2000, 26, 283.
[¹³] Zhu, H.; Bilgin, M.; Bangham, R.; Hall, D.; Casamayor, A.; Bertone, P.; Lan, N.; Jansen, R.; Bidlingmaier, S.; Houfek, T.; Mitchell, T.; Miller, P.; Dean, R. A.; Gerstein, M.; Snyder, M. Global Analysis of Protein Activities Using Proteome Chips. Science. 2001, 293, 2101.
[¹⁴] Ruiz-Talyor, L. A.; Martin, T. L.; Zaugg, F. G.; Witte, K.; Indermuhle, P.; Nick, S.; Wagner, P. Monolayers of derivatized poly(L-lysine)-grafted poly(ethylene glycol) on metal oxides as a class of biomolecular interfaces. Proc Natl Acad Sci USA. 2001, 98, 852.
[¹⁵] Mitchell, P. A perspective on protein microarrays. Nature Biotechnology. 2002, 20, 225.
[¹⁶] Gill, I.; Ballesteros, A. Bioencapsulation within synthetic polymers (Part A): sol-gel encapsulated biologicals. Trends in Biotechnology. 2000, 18, 282.
[¹⁷] Cho, E. J.; Tao, Z.; Tang, Y.; Tehan, E. C.; Bright, F. V. Tools to Rapidly Produce and Screen Biodegradable Polymer and Sol-Gel Derived Xerogel Formulations. Applied Spectroscopy. 2002, in press.
[¹⁸] Park, B. P.; Clark, D. S. Sol-Gel Encapsulated Enzyme Arrays for High-Throughput Screening of Biocatalytic Activity. Biotechnology and Bioengineering. 2002, 78-2, 229.
[¹⁹] Obert, R. and Dave, B. C. Enzymatic Conversion of Carbon Dioxide to Methanol: Enhanced Methanol Production in Silica Sol-Gel Matrices. J Am. Chem. Soc. 1999, 12192-12193, 121.
[²⁰] Taylor, S.; Croker, K. and Weber, S. Gel Pad Arrays and Methods and Systems for Making Them. PCT Patent Application No. WO 99/36576, Published Jul. 22, 1999.
[²¹] C. Lopreore, L. D. Byers. Arch. Biochem. Biophys. 349 (1998) 299.
[²²] A. K. Williams, J. T. Hupp, J. Am. Chem. Soc. 120 (1998) 4366.
[²³] J. D. Badjic, N. M. Kostic, Chem. Mater. 11 (1999) 3671.
[²⁴] S. A. Yamanaka, B. Dunn, J. S. Valentine, J. I. Zink, J. Am. Chem. Soc. 117 (1995) 9095.
[²⁵] S. A. Yamanaka, N. P. Nguyen, B. Dunn, J. S. Valentine, J. I. Zink, J. Sol-Gel Sci. Technol. 7 (1996) 117.
[²⁶] T. R. Besanger, Y. Chen, A. K. Deisingh, R. Hodgson, W. Jin, S. Mayer, M. A. Brook, J. D. Brennan, Anal. Chem. (2003) accepted.
[²⁷] L. Zheng, W. R. Reid, J. D. Brennan, Anal. Chem. 69 (1997) 3940.
[²⁸] S. Cesareo, S. Langton, FEMS Microbiol. Lett. 99 (1992) 15.
[²⁹] N. E. Dixon, P. W. Riddles, C. Gazzola, R. L. Blakeley, B. Zemer, Can. J. Biochem. 58 (1980)1335.
[³⁰] M. Zhou, Z. Diwu, N. Panchuk-Voloshina, R. P. Haugland, Anal. Biochem. 253 (1997) 162.
[³¹] M. Zhou, C. Zhang, R. H. Upson, R. P. Haugland, Anal. Biochem. 260 (1998) 257.

TABLE 1
Kinetic parameters for substrate turnover and enzyme inhibition for
free and entrapped enzymes and for enzyme microarrays.

| | GOx/HRP | | Urease/FD | | |
|---|---|---|---|---|---|
| | K_{M} (_M) | k_{cat} (s⁻¹) | K_{M} (mM) | k_{cat} (s⁻¹) | K_{I} |
| Solution | 103 ± 9 | 9 ± 1 × 10⁵ | 1.3 ± 0.2 | 78 ± 2 | 48-85^{a} |
| Entrapped | 188 ± 4 | 1.9 ± 0.3 × 10⁵ | 2.35 ± 0.03 | 1.33 ± 0.02 | 54 ± 2 |
| Enzyme in | | | | | |
| Platereader | | | | | |
| Microarray | 58 ± 3 | 4.9 ± 0.3 × 10⁴ | 1.9 ± 0.1 | 1.1 ± 0.1 | 62 ± 7 |

^{a}The range of K_{I} values is due to enzyme activity fluctuations at different pH values (5.5 to 8).

1. A microarray comprising one or more spots of a biomolecule-compatible matrix having two or more components of a protein-based system entrapped therein, wherein the one or more spots are adhered to a surface.

2. The microarray according to claim 1, wherein the biomolecule-compatible matrix is a sol-gel.

3. The microarray according to claim 2, wherein the sol-gel is prepared from one or more organic polyol silanes.

4. The micaroarray according to claim 3, wherein the organic polyol silane is derived from one or more of sugar alcohols, sugar acids, saccharides, oligosaccharides and polysaccharides.

5. The microarray according to claim 3, wherein the organic polyol silane is derived from one or more of allose, altrose, glucose, mannose, gulose, idose, galactose, talose, ribose, arabinose, xylose, lyxose, threose, erythrose, glyceraldehydes, sorbose, fructose, dextrose, levulose, sorbitol, sucrose, maltose, cellobiose, lactose, dextran, (500-50,000 MW), amylose, pectin, glycerol, sorbitol, and trehelose.

6. The microarray according to claim 5, wherein the organic polyol silane is derived from one or more of glycerol, sorbitol, maltose and dextran.

7. The microarray according to claim 3, wherein the organic polyol silane is selected from one or more of diglycerylsilane (DGS), monosorbitylsilane (MSS), monomaltosylsilane (MMS), dimaltosylsilane (DMS) and a dextran-based silane (DS).

8. The microarray according to claim 7, wherein the organic polyol silane is selected from one or more of DGS and MSS.

9. The microarray according to claim 2, wherein the sol-gel is prepared from one or more of functionalized or non-functionalized alkoxysilanes; functionalized or non-functionalized bis-silanes of the structure (RO)₃Si—R′—Si(OR)₃, where R may be ethoxy, methoxy or other alkoxy groups and R′ is a functional group containing at least one carbon; functionalized or non-functionalized chlorosilanes; silicates; and sugar, polymer, polyol or amino acid substituted silicates.

10. The microarray according to claim 9, wherein the sol gel is prepared from sodium silicate.

11. The microarray according to claim 1, wherein the matrix further comprises an effective amount of one or more additives.

12. The microarray according to claim 11, wherein the one or more additives are selected from one or more of humectants and protein stabilizing agents.

13. The microarray according to claim 12, wherein the one or more additives are selected from one or more organic polyols, hydrophilic, hydrophobic, neutral or charged organic polymers, block or randon co-polymers, polyelectrolytes, sugars and amino acids.

14. The microarray according to claim 13, wherein the one or more additives are selected from one or more of glycerol, sorbitol, sarcosine and polyethylene glycol.

15. The microarray according to claim 14, where the additive is glycerol.

16. The microarray according to claim 1, wherein the surface is a solid support made of glass, plastic, polymers, metals, ceramics, alloys or composites.

17. The microarray according to claim 16, wherein the surface is a solid support made of glass.

18. The microarray according to claim 17, wherein the glass is cleaned to substantially remove any organic matter and adsorbed metal ions.

19. The microarray according to claim 17, wherein the glass is modified with aminopropyltrithoxysilane (APTES), glycidoxyaminopropyltrimethoxysilane (GPS) or another suitable coupling agent that promotes adhesion of the microspots to the planar surface.

20. The microarray according to claim 19, wherein the glass is modified with glycidoxyaminopropyltrimethoxysilane (GPS).

21. The microarray according to claim 1, wherein the spots are spatially defined.

22. A method of preparing a microarray comprising:
(a) combining two or more components of a protein-based system with one or more biomolecule-compatible precursor solutions; and
(b) applying the combination of (a) to a surface in a microarray format.

23. The method according to claim 22, further comprising:
(c) allowing the combination of (a) to gel on the surface.

24. The method according to claim 23, wherein the two or more components of a protein-based system and one or more biomolecule-compatible precursor solutions are combined with an effective amount of one or more additives.

25. A method of performing multi-component assays comprising:
(a) obtaining a biomolecule compatible microarray comprising a matrix having two or more components of a protein-based system entrapped therein;
(b) exposing the biomolecule-compatible microarray to one or more test substances; and
(c) detecting a change in the protein-based system.

26. The method according to claim 25, further comprising comparing the change in the protein based system to a control, wherein a change in the protein based system upon exposure to a reagent of interest compared to the control is indicative of the effect of the test substance on the protein based system.

27. A kit, biosensor, micromachined device or medical device comprising the microarray according to claim 1.

28. A kit comprising one or more microarrays according to claim 1 and optionally, one or more of:
(a) reagents for use with the one or more microarrays;
(b) signal detection array-processing instruments;
(c) databases; and
(d) analysis and database management software;

29. A method of conducting a target discovery business comprising:
(a) providing one or more assay systems for identifying test substances by their ability to effect one or more protein based systems, said assay systems using one or more microarrays according to any one of claims 1-21;
(b) (optionally) conducting therapeutic profiling of the test substances identified in step (a) for efficacy and toxicity in animals; and
(c) licensing, to a third party, the rights for further drug development and/or sales or test substances identified in step (a), or analogs thereof.
